(19) United States
(12) Patent Application Publication
(10) Pub. No.: US 2010/0082085 A1
(43) Pub. Date: Apr. 1, 2010

(54) PORTABLE MOXIBUSTION ENERGY GENERATOR BY USING WAVELIKE TEMPERATURE VARIATION

(76) Inventors: Ching-Chang Pai, Taipei (TW); Ming-Hung Tsai, Taipei (TW)

Correspondence Address: Ching-Chang Pai, 235 Chung - Ho Box 8-24 Taipei, Taiwan, R. O. C., Taipei, 235, TW

(21) Appl. No.: 12/243,971

(22) Filed: Oct. 1, 2008

(51) Int. Cl. A61F 7/00; G05D 23/19

(52) U.S. Cl. 607/108; 219/241

(57) ABSTRACT

A portable moxibustion energy generator by using wavelike temperature variation comprises a box body; a heat unit; a temperature detection unit being connected to the heat unit; an operation unit installed within the box body and being electrically connected the temperature detection unit for determining a tendency of the heat generated from the heat unit; a circuit control unit electrically connected to the operation unit; and a power supply unit being electrically connected to the circuit control unit; the power supply unit supplying power to the heat unit for converting electric energy into heat energy. The temperature detection unit detects a temperature of the heat unit and determines a tendency of heat generated from the heat unit for determination; the heat unit supplying power dynamically so as to adjust the temperature of the heat unit. The method for performing the same is also included.

### FIELD OF THE INVENTION

The present invention relates to moxibustions and particular to a portable moxibustion energy generator by using wavelike temperature variation.

### BACKGROUND OF THE INVENTION

Acupuncture and Moxibutsion are important physical treatment ways in Chinese medical science. The acupuncture is to insert needles at the acupuncture points of human body so as to stimulate and have the effect of treatment. The Moxibutsion is to burn special Chinese mugwort on the acupuncture point to stimulate the acupuncture point by heat so as to adjust the physiological function from the flux of vital energies to improve the health of human body, cure and prevent diseases. The effect of the acupuncture and moxibustion treatment is quick. Thus the acupuncture and moxibustion treatment is widely used.

With the improvement of Chinese medicine, some scientific equipments, principles, and technologies may combined with acupuncture and moxibustion, such as electric, electronic, magnetic, laser, infrared, microwave, etc. These headways are used to stimulate the acupuncture points to have the affect treatment of human body.

The moxibutsion which is more related to the topics of the present invention is to heat special Chinese mugwort on the acupuncture point to stimulate the acupuncture point by heating and the drugs are also mixed thereto. Via the transfer of the heat energy pass into human body, it has the effect of disease treatment. The heat will stimulate skin and nerves continuously so as to adjust the immune system of human body. Achieve the purpose of treating the disease and preventing the disease. Therefore, in the prior art process of heat stimulation, a method for converting the electric energy into heat energy and the device of the same (called as heat moxibustion device) have been developed. However as the heat moxibustion device is used for a long duration, the skin will become blunt to the stimulation from moxibustion. Thus the function of the moxibustion will decrease gradually. It is necessary to increase the electric energy from the moxibustion device continuously so as to increase the stimulation and to increase the temperature on the skin. However the endurance of the skin to the heat energy is limited. Meanwhile, the temperature of the heat moxibutsion device is affected by the environment temperature so that the method is not effective.

Currently heat moxibustion device is used in Chinese medicine organization or physical treatment organization, which is inconvenient to be applied outdoor. Thus the patients are inconvenient, can not use the device whenever and wherever as needed. And narrow the benefit of the therapy.

### SUMMARY OF THE INVENTION

To achieve above object, the present invention provides a portable moxibustion energy generator by using wavelike temperature variation for treating the ache area of the patient without hurting the skin of the patient. Furthermore, the present invention is practical, without uncomfortable, safe, lower cost, good effect and has no side effect. The present invention can be applied to the acupuncture points of the patient to improve the circulation of blood and vital energy flow in the human body.

To achieve above object, the present invention provides a portable moxibustion energy generator by using wavelike temperature variation, comprising: a box body; a heat unit installed in the box body; a temperature detection unit installed in the box body and being connected to the heat unit; an operation unit installed within the box body and being electrically connected the temperature detection unit for determining a tendency of the heat generated from the heat unit; a circuit control unit installed within the box body and being electrically connected to the operation unit; a power supply unit installed within the box body and being electrically connected to the circuit control unit; the power supply unit supplying power to the heat unit for converting electric energy into heat energy; wherein the temperature detection unit detects a temperature of the heat unit and determines a tendency of heat generated from the heat unit for determination; the power supply unit supplying power dynamically so as to adjust the temperature of the heating unit. The present invention further provides a method for generating moxibustion energy in a portable device, comprising the steps of: using a power supply unit providing electric power to a heat unit for converting electric power into heat energy; using a temperature detection unit to detect whether a temperature of the heat unit is within a setting range of temperature; if yes, stop power supply to the heat unit; if no, the power is continuously supplied to the heat unit; determining a tendency of the heat generating of the heat unit; and power supply unit supplying power to the heat unit dynamically based on the tendency so as to adjust the heat generation of the heat unit.

In adjusting the heat generating of the heat unit, a transformation of the heat energy is detected and the tendency of the heat from the heat unit is determined; and electric power supplied to the heat unit is dynamically for adjusting the heat generating; the temperature of the heat energy increases from a lower temperature to a high temperature and then from a high temperature to a low temperature; and the heat energy is supplied from the heat unit based on portion of human body and a function of time to temperature.

Further the present invention causes that temperature increases from a low value to a high value and then from the high value to the lower value so as to form as a wavelike temperature performed. The advantage of the present invention is that the present invention can be directly applied to the pertinacious illness and pain place of the human body, alleviates the sour pain of the affected part immediately. This wavelike heating energy is released to the ache zone of the patient for a longer period so that expand the pores of the skin, improve the circulation system of blood, and the vital cells grow with a better effect. Thus, the present invention can treat disease effectively.

With the power of rechargeable battery, the wavelike temperature stimulation can performed for 10 hours. (Operating time of using is contingent upon environment) This wavelike temperature can be released for a longer course of treatment without skin injury of the high temperature, thus it is operated safely. The various objects and advantages of the present invention will be more readily understood from the following detailed description when read in conjunction with the appended drawing.

### BRIEF DESCRIPTION OF DRAWINGS

FIG. 1 is a perspective view showing the interior structure of the present invention.

FIG. 2 is a perspective view of the present invention.

FIG. 3 is a structural schematic view of the present invention.

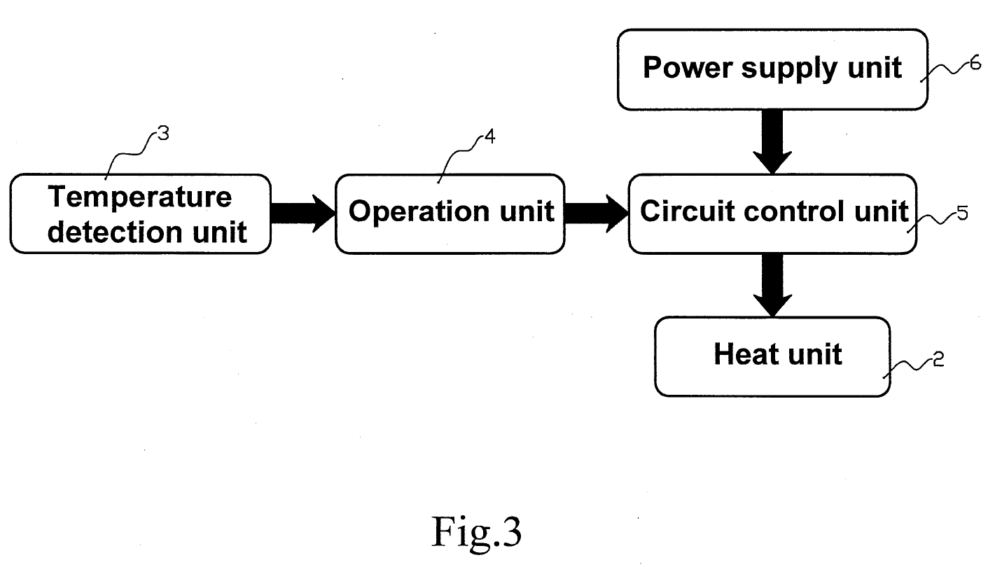

FIG. 4 is a schematic view showing the flowchart of the present invention.

FIG. 5 is a schematic view showing that the time period for supplying power is increased due to the body temperature descends rapidly.

FIG. 6 is a schematic view showing the time period for supplying power is increased due to the body temperature descends moderately.

FIG. 7 is a schematic view showing the time period for supplying power is increased due to the body temperature descends slowly.

FIG. 8 shows the relation of the power supply and the temperature of a portion of a human body in practical use.

### DETAIL DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

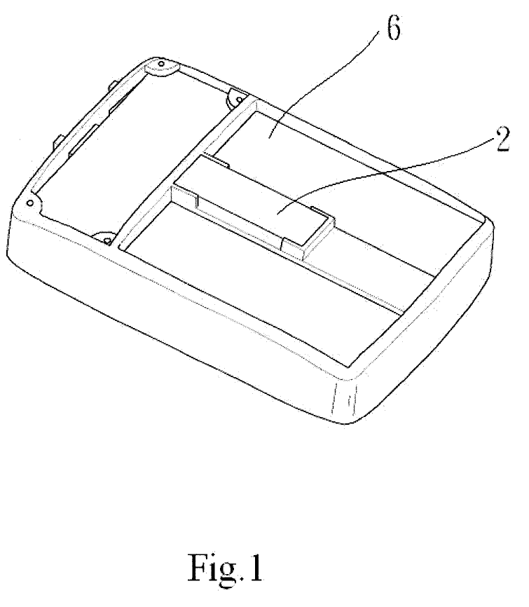

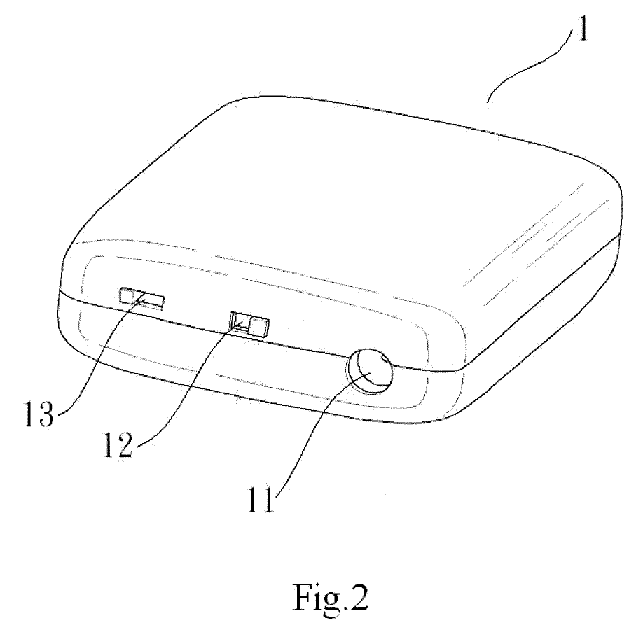

Referring to FIGS. 1 and 2, a portable moxibustion energy generator according to the present invention is illustrated. The present invention has the following elements.

A box body 1 is made of copper which is efficient in heat transfer. The box body 1 has a receptacle 11 for being connected with a DC power charger. The receptacle 11 is connected to a power supply unit 6 (for example a rechargeable battery). The box body 1 is installed with a control switch 12 and an LED indicating lamp 13. If necessary, the LED indicating lamp 13 can changed into a switch to control different temperatures.

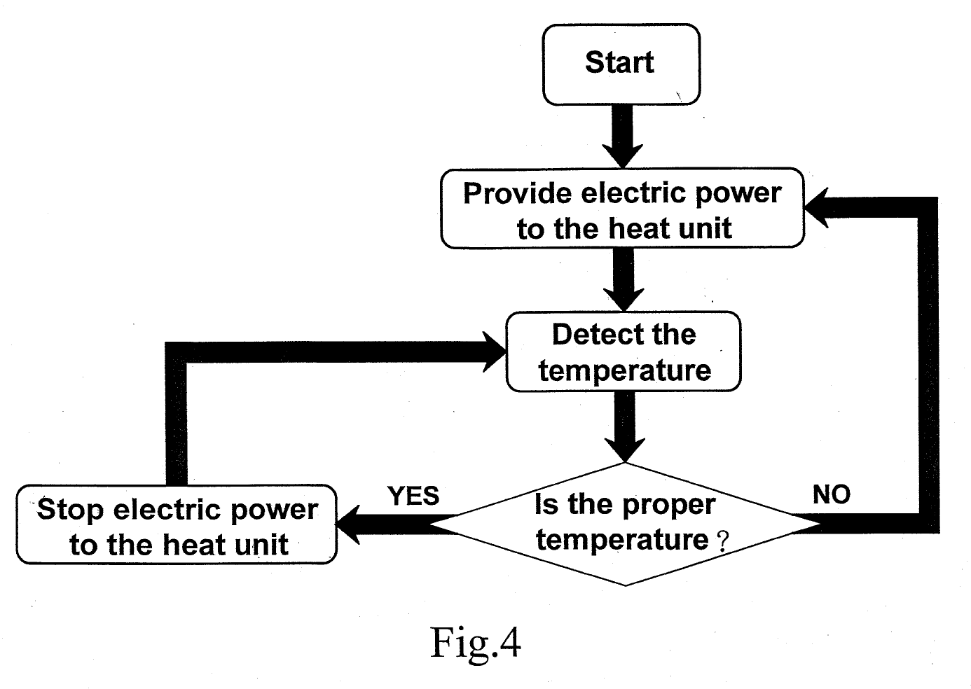

A heat unit 2 (referring to FIG. 4) is installed in an interior of the box body 1 and is adjacent to an inner wall of the box body 1 so that heat from the heat unit 2 can be transferred to the box body 1. The material of the heat unit 2 is selected from one of ceramic heat generating chip which is low-energy consumed, smokeless, odorless and environment-protected and safe. The rated voltage of the heat unit 2 is between 3 to 4.3 V with a rated current of 500 mA. The heat unit 2 will perform the temperature in a section to make people feel comfortable, for example about 43° C. so as to have the moxibustion effect. Moreover, the heat unit 2 of the present invention is heat generating chip made of carbon ceramics or made of silicon rubber or is by W (Wolfram), Mo (Molybdenum), Cr (Chromium) and Ni (nickel) alloy made.

A temperature detection unit 3 is installed in the interior of the box body 1 and is connected to the heat unit 2. The temperature detection unit 3 will detect the temperature of the heat unit 2. The temperature detection unit 3 has high heat conductive medium selected from Cu, Ag, Al, Au, Fe, YBCO super conductors. The heat conductive medium is installed within the temperature detection unit 3. Besides, the temperature detection unit 3 of the present invention is a thermistor formed by evaporated thin films, carbon or mixtures of carbon, or ceramic-like semiconductors formed by oxides of Cu, Mn, Mg, Ni, or U. Further, the temperature detection unit 3 may be a resistance temperature device (RTD) made.

An operation unit 4 is installed within the box body 1. The operation unit 4 is electrically connected to the temperature detection unit 3 for determining the heat generate tendency of the heat unit 2. The operation unit 4 may be a system on a chip (SoC) or a system on a Programmable chip (SoPC) which are a processor integrated with a plurality of IC units (including memories, CPU, I/O devices, etc.). The operation unit 4 may be an operating system device of 4 bits, 8 bits, 16 bits, 32 bits, 64 bits, etc. The operation unit 4 may be a differential comparators Chip.

A circuit control unit 5 is installed within the box body 1 and is electrically connected to the operation unit 4. The circuit control unit 5 of the present invention is a MOSFET (Metal Oxide Semiconductor Field Effect Transistor) or is formed by a Darlington circuit.

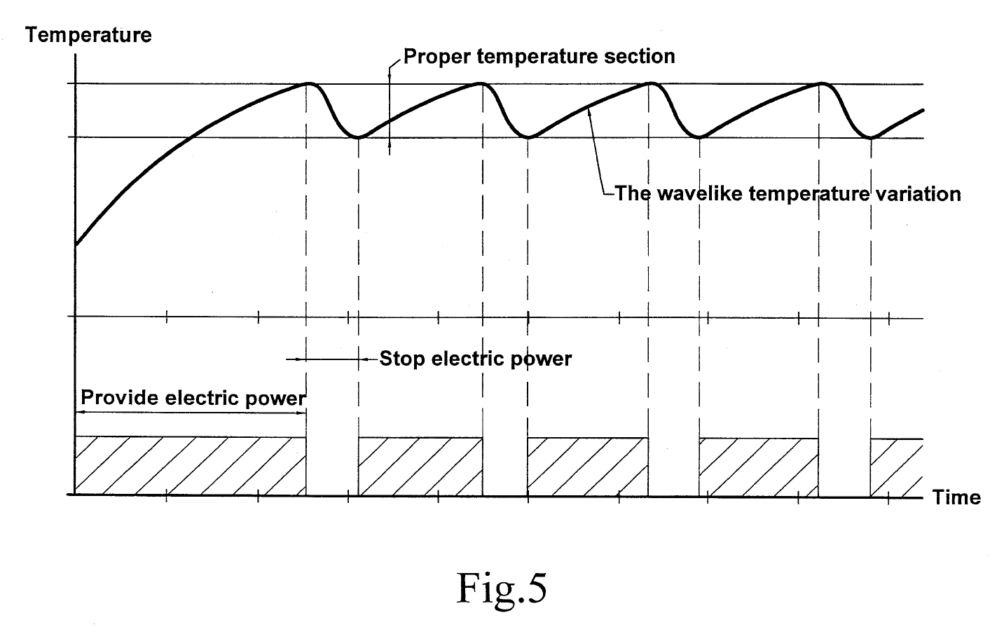

A power supply unit 6 is installed within the box body 1 and is electrically connected to the circuit control unit 5. The power supply unit 6 is power connected to the heat unit 2. The power supply unit 6 may be a battery set, for example a rechargeable battery set, even the battery is installed with overcharging and over discharging protection module. The battery may be a polymer lithium ion battery without memory effect and having a long lifetime. Or the battery is a NIH rechargeable battery, or a fuel cell, or others. In the present invention, the rechargeable battery is as a power source with low voltage working while it is safe in use and can be reused so that the cost is low. The over charging and over discharging protection module serves for monitoring the voltage and current of the battery set is charging and discharging. When the battery is over charged to a detection voltage of, 4.30±0.05V, the protection circuit will actuate to stop the charging circuit to have the function of protection the circuit. When the voltage of the battery set is descent to an over discharging detecting voltage, 3.0±0.10V, the protection module will actuate to stop the discharging circuit to have the function of over discharging protection. In the over current protection of the battery set, when short circuited or the discharging current is over the over current protection current, 3A, the protection module will actuate to stop the power offer so as to have the function of over current protection. In the present invention by above mentioned structure, as illustrated in FIG. 5, a temperature wave moxibustion energy generating method is provided. In that, the power supply unit 6 provides electric power to the heat unit 2 for converting electric power into heat power. The temperature detection unit 3 will detect the temperature of the heat unit 2 to determine whether the temperature is within a setting range (or value) of temperature, if yes, the power supply is stopped, if not, the power is supplied continuously. The tendency of the heat generation in the heat unit 2 is determined and electric power supply is dynamic so as to adjust the temperature of the heat unit 2.

While the power supply was stopped, the temperature of the heat unit 2 will go down gradually due to the contact with the human body or heat being absorbed in the ambient temperature. The cooling ratio of the heat unit 2 is positively proportional to the temperature difference between the heat unit 2 and the human body temperature or between the heat unit 2 and the ambient temperature. Assume T(t) is the temperature of the object (heat unit 2) at time t, when t=t₀, the temperature is T₀, assume H is the ambient temperature, meaning it is the temperature of the surrounding air.

### Newton's Law of Cooling is:

T′(t)=−α(T(t)−H)

Where α>0, which is a constant related to the object. It should be noted that the negative number in −α represents that when the temperature of the object is higher than the ambient temperature, the temperature of the object will descend (Cooling down). When the temperature of the object is lower than the ambient temperature, the temperature of the object will increase.

### Using Indefinite Integral:

$$\int \frac{{T}^{′} ( t )}{( T ( t ) - H )} d t = \int ( - α ) d t \\ ⇒ ln \vert T ( t ) - H \vert = - α t + c \\ ⇒ \vert T ( t ) - H \vert = C \cdot {e}^{- α t}$$

### Substitute T(t₀)=T₀, it Will Obtain:

T(t)=H+(T₀−H)e^{-α(t-t}^{₀}⁾

Therefore, in room environment, the temperature of the human body is related to the cooling rate of the heat unit 2. Thus, from the cooling rate of the heat unit, the variation of the human body can be got therefrom. In other words, the temperature detection unit 3 detects the temperature of the heat unit 2 at real-time. Meanwhile, the operation unit 4 and the circuit control unit 5 will calculate the cooling rate so as to get the wavelike temperature variation of human body so that the power supply unit 6 can supply power properly for increasing the temperature of the heat unit 2 which is related to the wavelike temperature variation of human body and the power supplied from the power supply unit 6.

Referring to FIGS. 5 to 8, in FIG. 5, it is illustrated that when the temperature of human body is low, the time period for supply power is increased. When the power supply unit 6 supplies power continuously, the human body will sense the increment of heat supplied. When the temperature increments to a limited value, the power supply unit 6 is stopped. Then the temperature of human body descends rapidly. Therefore, the power supply unit 6 can restore the power supply in a short time period (namely, the descending ratio is quick). Thus it can obtain that the power is supplied intermittently.

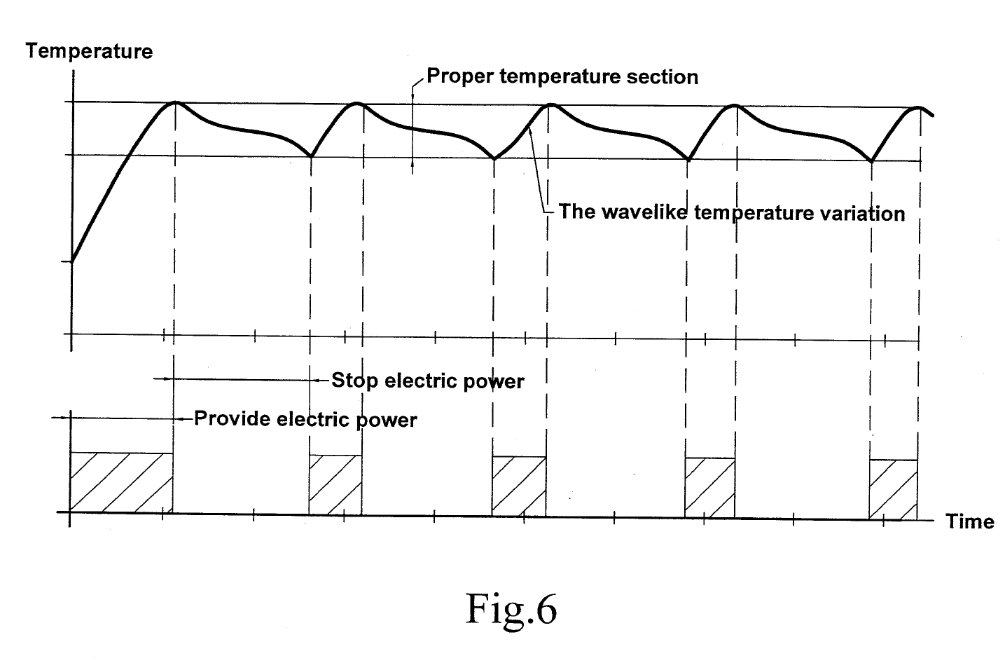

With referring to FIG. 6, when the temperature of human body is at a proper temperature and the power supply unit 6 supplies power continuously, the body temperature will respond to the increment of the heat. When the body temperature achieves to a limit value, the power supply unit 6 is stopped. At this moment, the human temperature descends with moderate ratio. And base on this status, the power supply unit 6 supplies the power with the duration of the moderate rate. The process can be repeated to have an intermittent power supply state.

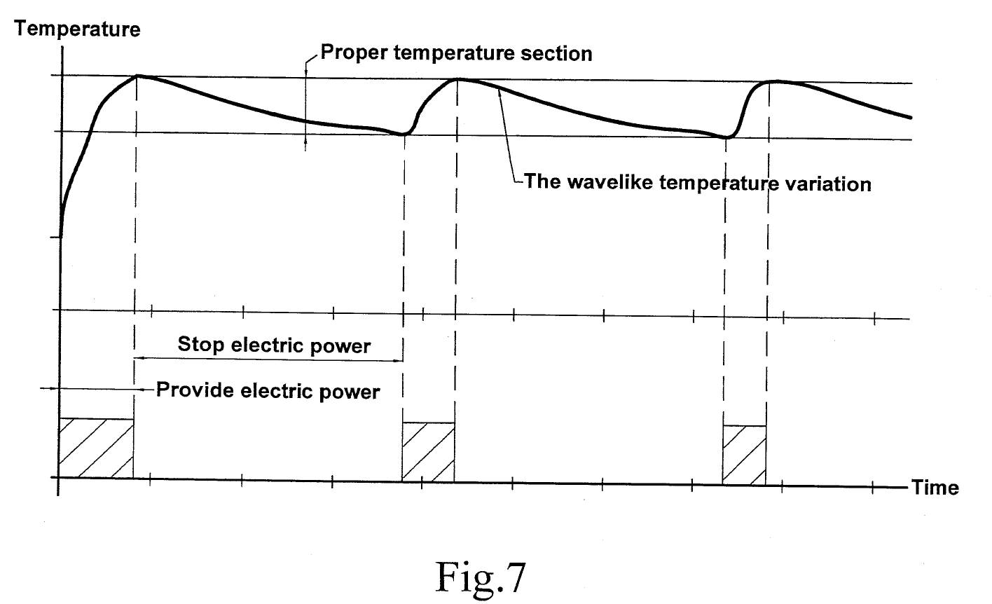

Referring to FIG. 7, the temperature of human body increases, the time period for supplying power will shorten. While the power supply unit 6 supplies power continuously. The body temperature will increase due to the sensation of the heat from the heat unit 2. When the body temperature increases to a limit value, the power supply unit 6 is stopped. At this moment, the body temperature descends slowly. As a result, the time period for restoration of the power supplied from the power supply unit 6 is prolonged (because the cooling rate is descending with a low speed). The process can be repeated to have an intermittent power supply state.

According to Chinese medicine, in the principle of heat transfer based on the function of the moxibustion, high temperature firstly applies upon the skin so as to stimulate the human nerve system and induce the vital energy in the sick portion and thus the physiological function of this portion of the body is activated. In the present invention, the temperature generated by the heat unit 2 is increased from a lower temperature to a high temperature and then descends from a high temperature to a low temperature. The energy is varied based on the requirement of the portion of the human body. The energy is applied to the sick portion of the body in a long time period so that expand the pores of the skin and facilitate the flow of blood to improve the circulation system and vital system of the body, also that the cells of the body will be enriched and the bad organizations in the body are repaired. Thus the disease is treated effectively.

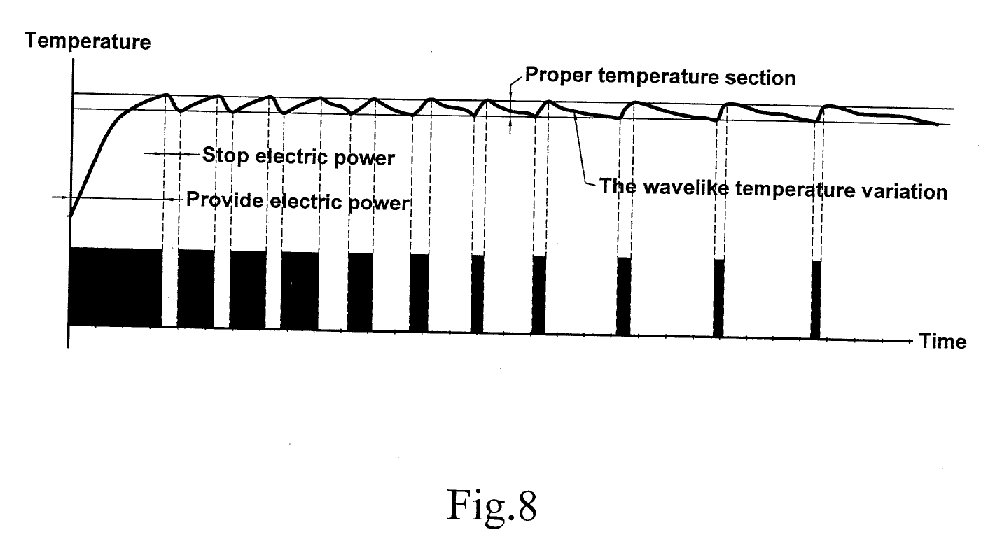

With referring to FIG. 8, in application of the present invention, the relation between the power supply and the body temperature is illustrated. The relation between the power supply from the power supply unit 6 and the body temperature at the portion of the human body is illustrated at the front section of the drawing, which is described with reference to FIGS. 5 to 7. After using for a time period, it is often that the physiological organizations of at the contacted location of human body have been activated so as to induce the cycle circulation of the temperature variation. Thus the power supply is decreased gradually. Besides, the present invention has the effect of power saving and prolonging the lifetime. This is the greatest technological difference between the present invention and general heaters.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A portable moxibustion energy generator by using wavelike temperature variation, comprising:
a box body;
a heat unit installed in the box body;
a temperature detection unit installed in the box body and being connected to the heat unit;
an operation unit installed within the box body and being electrically connected the temperature detection unit for determining a tendency of the heat generated from the heat unit;
a circuit control unit installed within the box body and being electrically connected to the operation unit;
a power supply unit installed within the box body and being electrically connected to the circuit control unit; the power supply unit supplying power to the heat unit for converting electric energy into heat energy; wherein the temperature detection unit detects a temperature of the heat unit and determines a tendency of heat generated from the heat unit for determination; so as to adjust the temperature of the heat unit.

2. The portable moxibustion energy generator by using wavelike temperature variation as claimed in claim 1, wherein a heat conductive medium of the temperature detection unit to the heat unit is a high heat conductive material selected from superconductors of Cu, Ag, Al, Au, Fe, and YBCO.

3. The portable moxibustion energy generator by using wavelike temperature variation as claimed in claim 1, wherein the heat unit is a ceramic heating chip.

4. The portable moxibustion energy generator by using wavelike temperature variation as claimed in claim 1, wherein the temperature detection unit is a thermistor sensor.

5. The portable moxibustion energy generator by using wavelike temperature variation as claimed in claim 1, wherein the temperature detection unit is a resistance temperature device (RTD).

6. The portable moxibustion energy generator by using wavelike temperature variation as claimed in claim 1, wherein the heat unit is polyester covered harter.

7. The portable moxibustion energy generator by using wavelike temperature variation as claimed in claim 1, wherein the heat unit is silicon rubber heater.

8. The portable moxibustion energy generator by using wavelike temperature variation as claimed in claim 1, wherein the heat unit is alloy lamina mixed W (wolfram), Mo (Molybdenum), Ni (nickel) or Cr (Chromium).

9. The portable moxibustion energy generator by using wavelike temperature variation as claimed in claim 1, wherein the operation unit is selected from one of a system on a chip (SoC) or a system on a Programmable chip (SoPC).

10. The portable moxibustion energy generator by using wavelike temperature variation as claimed in claim 1, wherein the operation unit is a differential comparator chip.

11. The portable moxibustion energy generator by using wavelike temperature variation as claimed in claim 1, wherein the circuit control unit is a metal oxide semiconductor field effect transistor (MOSFET).

12. The portable moxibustion energy generator by using wavelike temperature variation as claimed in claim 1, wherein the circuit control unit is an insulated gate bipolar transistor (IGBT).

13. The portable moxibustion energy generator by using wavelike temperature variation as claimed in claim 1, wherein the circuit control unit is a Darlington Circuit.

14. The portable moxibustion energy generator by using wavelike temperature variation as claimed in claim 1, wherein the power supply unit is one of the polymer lithium ion battery, the NiH battery, or the fuel cell.

15. A method for generating moxibustion energy in a portable device, comprising the steps of: using a power supply unit providing electric power to a heat unit for converting electric power into heat power; using a temperature detection unit to detect whether a temperature of the heat unit is within a set temperature range; if yes, no power is supplied to the heat unit; if no, power is continuously supplied to the heat unit; determining a tendency of the heat generation of the heat unit; and power supply unit supplying power to the heat unit dynamically based on the tendency so as to adjust the heat generation of the heat unit.

16. The portable moxibustion energy generator by using wavelike temperature variation as claimed in claim 15, wherein in adjusting the heat generation of the heat unit, a transferred of the heat unit is detected and the tendency of the heat from the heat unit is determined; and electric power supplied to the heat unit is dynamically for adjusting the heat generation; the temperature of the heat unit increases from a lower temperature to a high temperature and then from a high temperature to a low temperature; and the heat energy is supplied from the heat unit based on acting portions of human body and a function of time to temperature.
